(19) United States
(12) Patent Application Publication
(10) Pub. No.: US 2023/0255678 A1
(43) Pub. Date: Aug. 17, 2023

(54) AESTHETIC TREATMENT SYSTEMS AND METHODS

(71) Applicant: Revelle Aesthetics, Inc., Mountain View, CA (US)

(72) Inventors: Jonathan Podmore, San Carlos, CA (US); Arthur Ferdinand, Gilroy, CA (US); Alicia Holler, San Francisco, CA (US); Alejandro Garcia-Rubio, Berkeley, CA (US); Sam Rhea Sarcia, Lakeside, CA (US); Martin Leugers, San Francisco, CA (US); Tricia Wright, San Francisco, CA (US)

(21) Appl. No.: 18/137,018

(22) Filed: Apr. 20, 2023

Related U.S. Application Data
(63) Continuation-in-part of application No. 17/154,232, filed on Jan. 21, 2021.
Application No. 17/154,232 is a continuation-in-part of application No. PCT/US2019/042871, filed on Jul. 22, 2019.
(60) Provisional application No. 62/702,314, filed on Jul. 23, 2018.
(60) Provisional application No. 62/736,016, filed on Sep. 25, 2018.
(60) Provisional application No. 62/798,515, filed on Jan. 30, 2019.
(60) Provisional application No. 62/802,368, filed on Feb. 7, 2019.
(60) Provisional application No. 62/825,447, filed on Mar. 28, 2019.

(51) Int. Cl. A61B 18/14

(52) U.S. Cl. CPC A61B 18/14; A61B 2018/1422; A61B 2018/00464

(57) ABSTRACT

Systems and methods for aesthetic treatment of skin including an apparatus that applies or a method involving separating tissue to eliminate or reduce the appearance of unwanted features. In one approach, an interventional tool is placed between tissue layers to engage and treat connecting tissue layers.

### CROSS-REFERENCE TO RELATED APPLICATIONS

This application is a continuation-in-part of U.S. application Ser. No. 17/154,232, filed Jan. 21, 2021, which is a continuation-in-part of International Application Serial No. PCT/US19/42871, filed Jul. 22, 2019, which claims priority to each of U.S. Application Ser. No. 62/825,447, filed Mar. 28, 2019, U.S. Application Ser. No. 62/802,368, filed Feb. 7, 2019, U.S. Application Ser. No. 62/798,515, filed Jan. 30, 2019, U.S. Application Ser. No. 62/736,016, filed Sep. 25, 2018, and U.S. Application Ser. No. 62/702,314, filed Jul. 23, 2018, the entire disclosures of each of which are expressly incorporated herein by reference.

### FIELD OF THE DISCLOSURE

The present disclosure generally relates to aesthetic treatment systems and methods.

### BACKGROUND OF THE DISCLOSURE

There is a continuing need for an effective approach to improving the appearance of the surface of skin. In one or more approaches, it is desirable to treat cellulite, also known as gynoid lipodystrophy, nodular liposclerosis, edematofibrosclerotic panniculopathy, panniculosis, adiposis edematosa, demopanniculosis deformans or status protrusus cutis. Additionally, there is a need for treatment devices for use in scar release, acne subcision, facial fold and/or facial lift procedures. Moreover, there is a need for proactive treatment modalities that prevent future or reoccurrence of skin discontinuities and which are easy and effective to use.

It has been reported that more than 85% of women have cellulite thus suggesting that cellulite is a physiologic rather than pathologic condition. The existence of fat in the reticular dermis alone is not thought to cause cellulite. Cellulite can be described as the herniation of subcutaneous fat within fibrous connective tissue that is expressed as dimpling of the skin. This fat loading can lead to stress on connective tissue located between fat lobules. Such dimpling is more common in women than men due to the orientation of subcutaneous fibrous structures defining chambers containing fat cells. In fact, it is this structure that is believed to cause the appearance of cellulite more than being overweight. Often, cellulite appears on the pelvic region including the buttocks, lower limbs and abdomen.

Subdermal fat layers below the epidermis are contained between dermal layers connected by septa which act as connective tissue between the dermal layers. In men, the septa are arranged more randomly and densely oriented in a more criss-crossed configuration while the septa in women are generally more parallel in arrangement. Also, men have thicker dermis and more angled septa relative to the skin surface whereas women have relatively thinner dermis which thins with age, and septa that are perpendicular to the skin surface. Moreover, women with cellulite have exhibited thickening of the septa in the regions of cellulite and tensioning of septa highlights cellulite. In women, fat storage in adipose tissue has a biological purpose in that it is maximized ensuring adequate caloric availability for pregnancy and lactation. An increase in fluid retention or proliferation of adipose tissue in such subdermal fat layers can further result in the appearance of cellulite where the septa is maintaining a first distance between dermal layers, thus creating dimples, whereas pockets between septa bulges. Over time, the septa may stretch, then eventually contract and harden thus retaining tissue layers at fixed distances, but pockets between such septa may be expanded thus adding to the appearance of cellulite.

Various approaches have been taken to treat or address cellulite. Early treatments involved attempts at increasing circulation and fat oxidation in areas exhibiting cellulite. Here, substances such as hyaluronic acid and aminophylline were injected in the target areas to reduce cellulite. Other approaches involved electroporating the target areas followed by the application of mesotherapy, or applying dermological creams or other supplements to cellulite. These approaches could be supplemented by massage or massage was used alone for the purpose of promoting increased fat reabsorption or drainage of fluids and toxins in the treated areas. Ultrasound has also been proposed to disrupt subcutaneous tissues and fat and has been used in combination with liposuction. Low acoustic pressure in combination with the infiltration of microbubbles has also been employed to reduce the appearance of cellulite, as has the use of other energies such as lasers and radio frequency. Such approaches have been characterized by limited or unpredictable results. More recently, the cutting of septa with blades or needles in the subdermal region has been employed. Prior approaches have been found to be labor intensive and very traumatic to the tissue leading to numerous skin entry sites, bleeding, bruising, tough tissue nodules, long, painful recoveries and inconsistent results.

There is also a significant need for devices to treat scars and acne, or for use in facial fold and/or facial lift procedures. In conventionally available procedures, treatment devices are required to be inserted within tissue close to the interventional site, and travel within tissue to the site results in cutting or disrupting non-target tissue. There is thus a need for a treatment device that can be placed into tissue at more cosmetically desirable locations where the treatment device includes a blade that is only exposed once it reaches the treatment tissue. In addition, there is benefits associated with using a single entry site to treat multiple target locations through the single entry site since safe and minimally traumatic navigation between entry and the targeted tissue is desirable.

Accordingly, there is a need for effective and efficient approaches to improving the aesthetic appearance of skin including treating, minimizing or eliminating trauma. These approaches should be associated with predictable results and be relatively easy to employ.

The present disclosure addresses these and other needs.

### SUMMARY OF THE DISCLOSURE

Briefly and in general terms, the present disclosure is directed towards aesthetic treatment systems and methods involving an apparatus that facilitates and methods involving, depending on the system used and force applied by the user, stretching, re-orienting, disrupting, cutting, slicing, and/or tearing tissue. In one aspect, the treatment approach involves a tissue cutting or slicing system. In another aspect, tissue cutting or slicing is combined with disruption and/or localized removal of fat.

In one embodiment, a cellulite treatment device is mounted at a distal end portion of a shaft and is sized and shaped to be advanced between tissue layers. In one particular aspect, fibrous septa that connect superior and inferior fascia plateaus within skin can be crossed with the treatment device using one or more of an array of tools to engage, and depending on the tool used and force applied by the user, stretch, re-orient, tear, disrupt, cut or slice septa. By doing so, the target subcutaneous connective tissue associated with the surface defect can be directly modified with minimal impact to surrounding blood vessels and lymphatic system and fat can be more evenly distributed and skin can assume a smoother appearance.

In various aspects, a handle assembly is provided to actuate a treatment device. In one or more approaches, the handle assembly includes an actuatable assembly that controls configuring a treatment device into one or more of home or sheath, open or hook, and active or cut configurations. The actuatable assembly can include one or more of a slider, an active or cut button and a return home button. Alternatively, the handle assembly includes structure that facilitates an automatic return to home upon completion of stretching, re-orienting, tearing, disrupting, cutting or slicing of septa or other tissues.

In one or more aspects, a cellulite treatment system embodies a tool facilitating an ability to reach and treat all target cellulite appearance areas through a single skin entry on each side of the patient or a limited number of entries through the skin. In certain aspects, such tool is sized, shaped and configured (e.g. less than or equal to about three millimeters diameter, more preferably less than or equal to about 2 millimeters, and blunt dissection tip) to be placed within and advanced between tissue layers on its own and without assistance from external skin stabilizing structure, such as a suction device. Entry points through the skin such as high on the hip under where a bikini or underwear strap would be and along creases or transitions between buttocks and thighs (for example, gluteal crease) are employed. Identification and assessment of target septa is accomplished by pushing, pulling or otherwise tensioning septa in areas believed to be associated with the appearance of cellulite on the outside of skin. It has been recognized that septa causing a dimple or depression are located at various angles and locations relative to the dimple or depression observed on the skin and are not necessarily directly below such appearances of cellulite, and the treatment system and method is configured to identify the septa responsible for the appearance of cellulite that has been marked on the skin and target treatment on those septa and leave adjacent septa, blood vessels, etc. intact. Moreover, a range such as a small subset or a larger number of septa can be the structure causing a particular depression or dimple.

In one method, anesthetic is injected into the treatment site transcutaneously or subcutaneously, a cellulite treatment system is inserted subcutaneously across the treatment site and used to identify the septa responsible for a depression or dimple by pushing or pulling on various septa to cause a depression in the skin in the target area, and a cutting or slicing device or septa disruption structure is placed subcutaneously at the treatment site and employed to engage and cut or slice or break the septa tissue. In one particular aspect, the patient is directed to clench their buttocks and/or leg muscles to help facilitate identifying target areas and after septa treatment confirm release of septa that create dimples or depressions. Alternatively, the physician can press in a cranial to caudal direction on the skin above the treatment target or pull from below the treatment target. Remote imaging or ultrasonic or fluoroscopic energy can be employed to observe the procedure. A resizing or alternative configuration of the treatment structure can be employed to complete the treatment of a particular area. The treatment device is then repositioned to treat additional areas. The treatment device can be configured to treat a plurality of areas simultaneously or in succession without removing from the patient or a spot treatment approach can be taken. Additionally, through one or more entry points, various treatment trajectories are directed and in certain applications a steerable introducer is used to access treatment areas. Further, anti-inflammatory, collagenase, deoxycholic acid, salicylic acid, glycolic acid, hyaluronic acid, tranexamic acid or cellulite treatment medicants can be employed at the interventional site separately or directly by the interventional device or other procedural instrumentation. Aspects of the current invention include specific identification of the septa responsible for the cellulite appearance, severing or separation of those septa, confirmation intra-operatively that the separation of those septa was accomplished and the prevention of the re-appearance of the cellulite.

In various aspects, the treatment device can include one or more of cutting structure that cooperates with a septa or connective tissue hooking or engaging element to one or more hook or engage then cut, slice, tear or disrupt septa or connective tissue. One or more of the septa or connective tissue hooking element and the septa or connective tissue cutting element is convertible from a hooking configuration to a cutting configuration and from a cutting configuration to a hooking configuration or to a stored configuration. The treatment device can further include various approaches to blocking structure that selectively blocks one or more cutting elements. In another particular approach, the treatment device is embodied in an elongate member insertable through the skin capable of expanding at least one region from a smaller state to a wider state, and when in the wider state is configurable to both hook and cut, slice or disrupt target septa or connective tissue. In one specific approach, a treatment device is embodied in an assembly including an elongate member that is configurable to present a hooked cutting structure for use in treating cellulite, scar or connective tissue existing in tissue planes, and includes a damper that reduces noise and slows the return of the device to a home configuration.

The cellulite treatment system also involves in certain approaches, illumination such as a bright light configured at or emitted through a tip of treatment structure or placed along or at strategic locations along treatment structure for the purposes of tracking advancement of the tool to the treatment site and locating intra-dermal structures at the treatment site. In this way, direct observation of the treatment device by transillumination through the skin is provided and positioning, depth and performance thereof subcutaneously is readily available to an operator.

In one or more further aspects, the disclosed devices can be used in scar release, acne subcision, facial fold and/or facial lift procedures, or in any area where tissue disruption is needed in the subcutaneous space between tissue layers of the body. Transillumination can be used or specific devices or systems can lack transillumination structures or assemblies. In various approaches, a treatment device can be placed into tissue at more cosmetically desirable locations where the treatment device includes a blade that is only exposed once it reaches the treatment tissue. In addition, a single entry site can be used to treat multiple target locations through the single entry site since safe and minimally traumatic navigation between entry and the targeted tissue is possible.

These and other features of the disclosure will become apparent to those persons skilled in the art upon reading the details of the systems and methods as more fully described below.

### BRIEF DESCRIPTION OF THE DRAWINGS

FIGS. 1A and B are perspective views, depicting cellulite on a subject's skin and a plan for treating the cellulite.

FIG. 1C is a top view, depicting treatment of a subject lying on a treatment table.

FIG. 1D is a top view, depicting a cellulite treatment assembly and approach for treating cellulite.

FIGS. 1E-G are side views, depicting the complex anatomy of septa.

FIGS. 2A-G are partial cross-sectional views, depicting one embodiment of treating septa below a skin surface.

FIGS. 3A-C are perspective, side and cross-sectional views, depicting one preferred embodiment of a treatment system.

FIG. 4A is a perspective view of a cellulite treatment assembly;

FIG. 4B is a right side view of the assembly of FIG. 4A;

FIG. 4C is a left side view of the assembly of FIG. 4A;

FIG. 4D is a rear side view of the assembly of FIG. 4A;

FIG. 4E is a front side view of the assembly of FIG. 4A;

FIG. 4F is a top side view of the assembly of FIG. 4A;

FIG. 4G is a bottom side view of the assembly of FIG. 4A;

FIG. 4H is a perspective view of another cellulite treatment assembly;

FIG. 4I is a right side view of the assembly of FIG. 4H;

FIG. 4J is a left side view of the assembly of FIG. 4H;

FIG. 4K is a rear side view of the assembly of FIG. 4H;

FIG. 4L is a front side view of the assembly of FIG. 4H;

FIG. 4M is a top side view of the assembly of FIG. 4H;

FIG. 4N is a top side view of the assembly of FIG. 4H;

FIGS. 4O-Q are cross-sectional and side views, depicting components of the handle assembly of FIGS. 14A-C.

### DETAILED DESCRIPTION OF THE DISCLOSURE

Before the present systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described.

It must be noted that as used herein and in the appended claims, the singular forms “a”, “an”, and “the” include plural referents unless the context clearly dictates otherwise. Thus, for example, reference to “the system” includes reference to one or more systems and equivalents thereof known to those skilled in the art, and so forth.

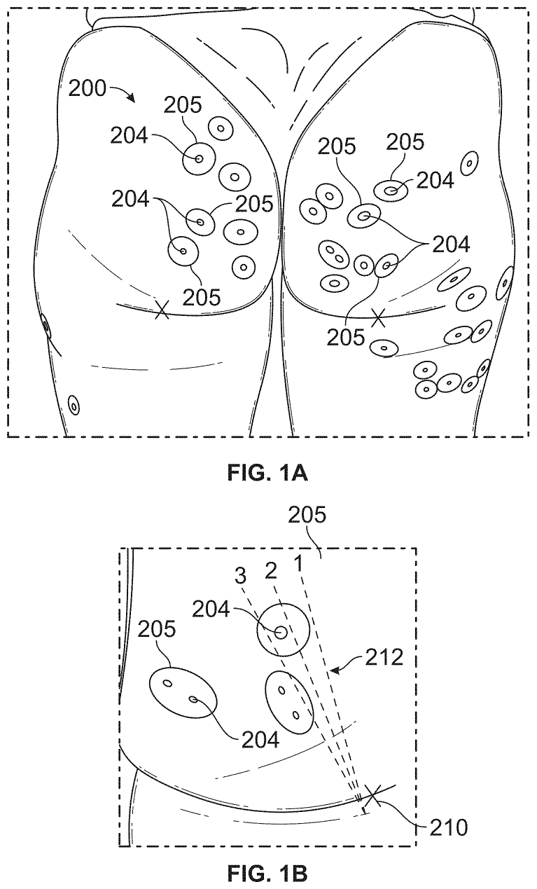

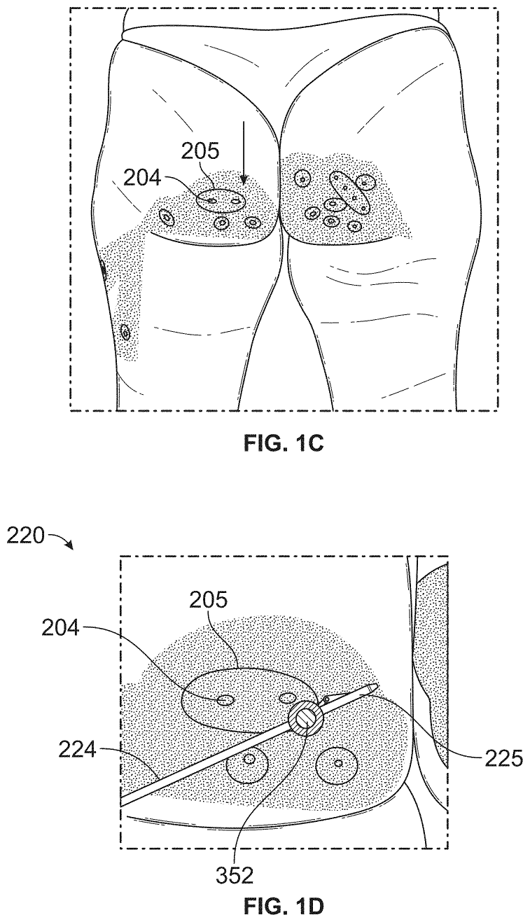

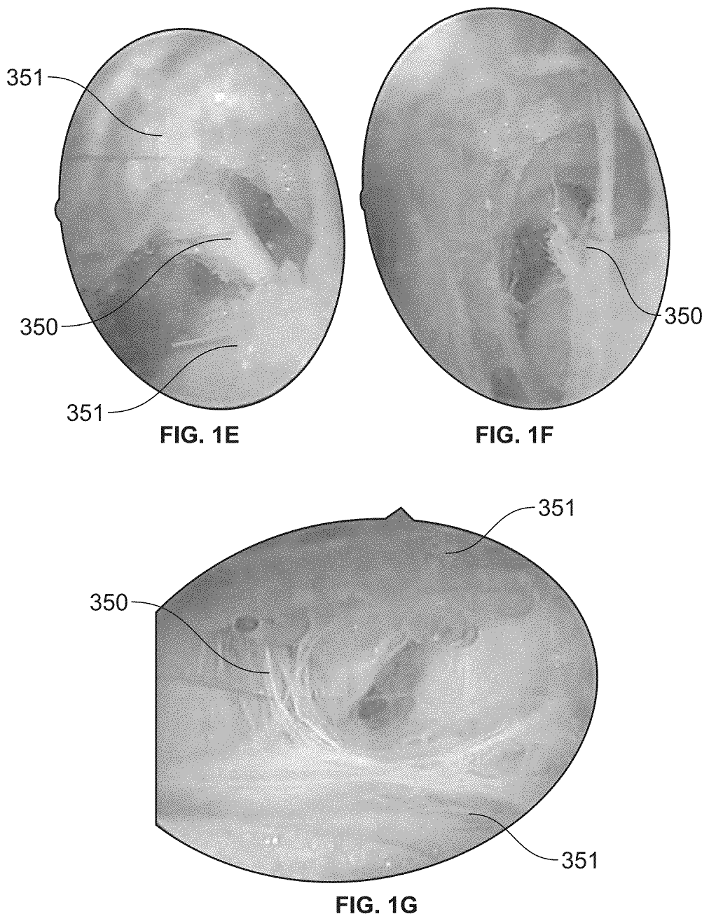

With reference to FIGS. 1A-B, there is shown a person exhibiting cellulite 200 about their thighs and buttocks. In one approach to treatment, dimples, linear depression and/or other depressions characteristic of the cellulite 200 intended to be treated are identified or circled with a marker to mark or outline edges 204 of depressions, preferably while the patient is standing as for most patients the appearance of their cellulite disappears when they lie down on their stomach because gravity is pulling in a different direction. The patient can be asked to lunge or clench tissue to aid in the identification of treatment areas. Approximately an 8 mm margin area 205 around targeted dimples, linear depressions or other depressions is marked on a patient's skin to identify an area for the physician to check for fibrous septa that are responsible for creating the dimple, liner depression or other depression. Such margins can assume various shapes dictated by the cellulite or depressions formed on a patient's anatomy and for example, can be defined as circles, ellipses or D-shaped treatment margins and the margins can encompass one or more targeted areas. In another embodiment, computerized imaging equipment is used to locate and mark dimples and/or depressions. In FIGS. 1A-B, dimples and depressions are marked for possible treatment. The physician treating the patient determines an instrument insertion site 210 and paths 212 that most efficiently treat cellulite with a minimal amount of insertion sites and instrument paths under the skin. Preferably, an instrument insertion site is chosen that is in a crease or fold of skin such as where the buttocks meets the thigh (i.e., gluteal crease) or in the crease between the two buttocks at a location that is not seen when the buttocks are in natural contact for improved cosmesis after the procedure healing period. Each depression can be divided into 6 mm lanes. With drawn 8 mm margins 205, each depression will thus have at least three lanes. Notably, multiple septa contribute to each depression. In certain patients, the inner thigh is chosen as an insertion site as this location is less visual as it heals, or the lateral thigh region or superior buttocks are used as alternative or additional insertion sites. Such treatment paths are selected by the operator preferably using a straight edge that bends or contours to the patient or can be generated automatically by employing a computerized controller programmed to most efficiently address and measure cellulite residing in a pre-defined treatment site. Each depression is treated from right to left since the treatment device deploys to the left and therefore treating from right to left makes it easier to keep track of which lanes have been treated. In various approaches, a treatment plan can involve treating sites in one or more of a counter-clockwise, right to left, lateral to medial or proximal to distal order. Once all lanes have been treated, an entire depression is verified from right to left and treated depressions are then marked with a surgical marker as being treated after verification. The computerized controller can be associated with a scanner that identifies specific dimples and areas for treatment such as by employing laser technology. In this regard, the computerized controller includes a program specific to cellulite treatment and is used in conjunction with an electronic and mechanical device and comprises or includes a non-transitory computer-readable storage medium and a computer-program mechanism embedded therein to both identify treatment areas and to plot primary and alternative approaches to treatments. In another embodiment, computerized visualization and treatment planning equipment is used to assist the physician in determining insertion site locations and paths to be taken to the marked targets.

Once a treatment approach is planned, the patient lies down on their stomach on the treatment table. Alternatively, because of the minimally invasiveness of the current approach, a patient can be treated while standing, particularly for a small number of treatment targets, or while standing and leaning forward on a support and alternatively between standing and leaning forward so that gravity can help identify and confirm treatment of the targeted septa. The patient can also be asked to lunge or clench muscles to aid in identifying treatment sites. Moreover, the measurement device creates a complete three-dimensional map of all cellulite relative to normal skin. By dating and comparing improvement of volume of divots or dimples versus normal idealized surfaces, the operator calculates total and local volume benefits of therapy and track improvement over time.

In one specific approach, as shown in FIG. 1C, anesthesia (represented by the shaded area) is thoroughly delivered subcutaneously beyond marked areas (approximately 1.5 inches) so as to provide room for a distal end of the treatment device 225 within the anesthetized area (see FIG. 1D). In one approach, multiple treatment targets along lines are treated from a single entry 210 at the lateral end of the gluteal crease (FIG. 1B). Thus, treatment can be directed to treat targeted septa, or additionally or alternatively, treatment can be transverse, preferably perpendicular, to the linear depressions on the posterior thigh or lateral thigh as shown in FIG. 1C for illustrative purposes to treat targeted septa. Treatment can be directed at various positions about connecting tissue or septa. That is, septa can be engaged, stretched, re-oriented, torn, cut, sliced, ruptured or disrupted from various sides or angles respecting septa. Thus, septa can be treated from above, below or the sides of septa to achieve the best results. For example, in a particular situation, treatment can be most effective from above a particular connecting tissue to take advantage of gravity where treatment forces placed on the connecting tissue coincide with the direction of gravity or the direction that gravity most often works on a standing body, as it has been observed that cellulite is often most visible in a standing individual. Additionally, using a limited thigh line treatment, the entirety of underlying septa associated with a linear depression in skin are not disrupted or treated but rather treatment involves approaching the linear depression perpendicular to or at an angle thereto and just a portion of the underlying septa is released. The treatment instrument is re-sheathed and repositioned slightly such as a few millimeters thereby leaving an area of undisrupted or treated septa, followed by disrupting or treating additional septa and subsequent re-sheathing, repositioning and disrupting or treating further septa as deemed necessary.

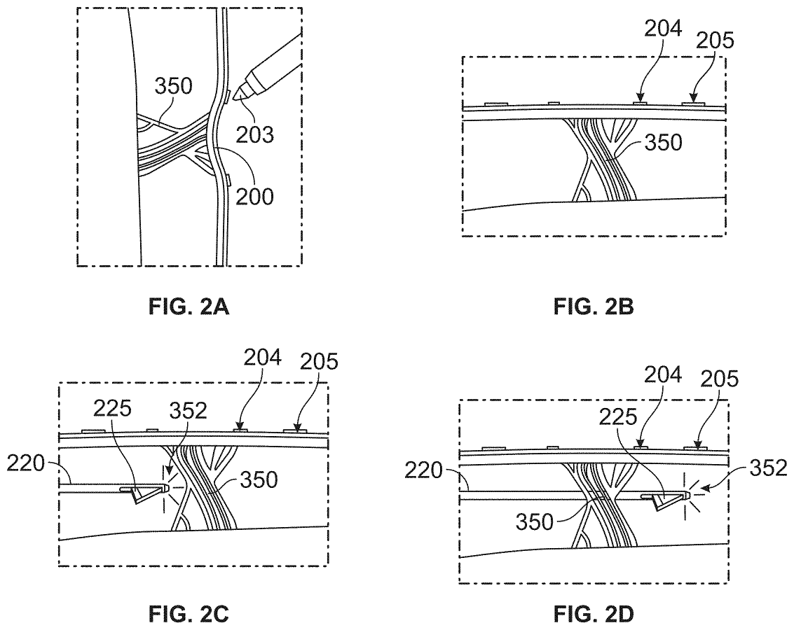

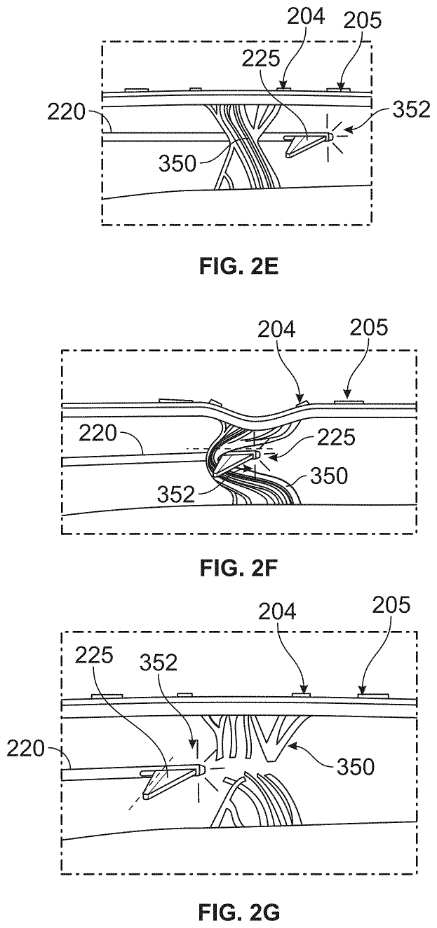

Turning now to FIG. 1D, there is shown a cellulite treatment assembly 220 and an elongate member or needle-sized structure preferably three millimeters or less, more preferably two millimeters or less, in diameter, like structure 224 extending longitudinally therefrom. A force gauge (electronic or mechanical) can be provided to ensure that a pre-determined amount of force would be applied to the tissue when testing the septa to prevent over or under pulling. A treatment device 225 capable of one or more of engaging, stretching, slicing, cutting or disrupting connective tissue is configured at a distal end portion of the elongate member 224 (e.g., FIGS. 2A-G). A light source 352 is provided proximal of the treatment device 225 to assist in guiding the treatment device 225 to targeted treatment locations. The treatment device can define an aesthetic treatment assembly including an elongate member extending from a handle and configured to be insertable through the skin and a mechanical septa disrupting element attached to a distal end of the elongate member that is capable of expanding at least one region from a smaller state to a wider state and engaging one or more regions of septa subcutaneously, wherein the mechanical septa disrupting element is configurable to both define septa hooking structure as well as septa severing or disrupting structure. All cutting means can be combined with or further energized with RF, a laser, ultrasonic or thermal energy to produce cutting and coagulation together or separately. Moreover, the cutting means can include a blade that is one or more of highly sharpened, hardened or coated (for example titanium nitride or Teflon).

In certain aspects, there can be a single entry site or two entry sites on each side of the patient, one high on the hip and another along the crease or transition between the buttocks and thigh, or at the inner thigh. Such locations are characterized in that they can be easily hidden either naturally or by clothing. Treatment targets, depressions and dimples that have been marked on the skin surface while the patient is standing often go away when the patient lies down on their stomach because gravity acts on the skin and underlying connective tissue in a different direction such that the ink mark 204 is apparent, but the dimple or depression is not. The disclosed interventional devices are configured such that a user can approach a target location and first use the interventional device to push, pull or otherwise tension septa in a target area under the skin to identify the specific septa impacting the target and/or which is the cause of the appearance of cellulite. In other words, pulling or pushing is performed on the septa under the skin to find the one(s) that create the dimple or depression in the skin surface. Notably, enough force is employed in pulling or pushing septa to create a dimple or depression on skin and an assessment is made to determine if the created dimple or depression corresponds to targeted dimples or depressions that have been marked for treatment. If so, then the engaged septa are treated as described herein and this approach is repeated for all targeted treatment areas. The operator also confirms that all the septa associated with a targeted dimple or depression have been treated with the treatment device so that all of the septa associated with targeted dimples or depressions have been completely released.

With reference to FIGS. 1E-G, it has been recognized that septa 350 define a complex network of connections between tissue layers 351 under the skin in and about the cellulite target areas, and septa includes “webs”, “trunks” and “branches from trunks” which connect tissue layers. It has also been recognized that septa can be quite elastic, stretching as much as about 10-20 mm before it creates depressions associated with the targeted area. In other circumstances, septa are inelastic and distances of about 10-25 mm are involved in creating enough tension for a dimple or depression to appear on the skin because of needed length of movement to tension long septa. Verification that all septa associated with a treatment target have been cut thus can involve multiple passes within a target area to ensure that an entire network of septa have been cut. Special attention is given to secondary septa that can be difficult to identify until after more readily identifiable or primary septa are cut. Secondary septa are septa that cause a shallower or smaller depression than the primary septa. Shallower or smaller depressions are not as noticeable on the skin when a patient is standing but after the primary septa to have been cut and there is no more deep depression, the shallower or smaller depression caused by the secondary septa becomes much more noticeable. Moreover, secondary septa and patterns of septa are expected and consequently searched in areas where there are multiple or closely located appearances of cellulite. In this way, a precise approach to relieving connective septa is taken in that septa hooked that create a depression outside the marked treatment area are released uncut and septa associated with targeted cellulite are cut. By taking multiple precision passes under a target area, all cellulite-forming septa, primary and secondary, are treated.

For some treatment targets, taking an approach from an entry located inferior the treatment target, advancing the end of the interventional device beyond the treatment target and then pulling inferiorly (effectively the “down” direction if the patient was standing) can provide a better approach, for example, for treatment targets on the leg, to re-create the dimple when the patient is lying down. One or more strain gauges can be incorporated within the treatment device to help identify target septa as well as to assess the progress and completion of treating septa. This facilitates targeting of key septa in a less impactful way, ideally minimizing bruising or other issues associated with cutting or disrupting a large area around the target. There are thus herein shown various approaches to treating cellulite expressed as dimples or depressions 200 in the skin surface. Moreover, the handle portion can be employed to create an indentation in skin through which interventional devices can be inserted subcutaneously. A treatment regimen is selected for inserting interventional instruments based upon the subject's anatomy as it relates to the septa 350 connecting tissue layers that define the chambers retaining fatty or other tissues. If desired, while anesthetic and/or sedation is taking effect, ultrasound can be used to assess the subcutaneous trajectory and depth of the various connective tissue bands responsible for the surface unevenness. The ultrasound evaluation can help with the particular trajectory selected for the desired depth. The ultrasound evaluation can also help with positioning the distal end portion of the treatment instrument strategically at the connection point between the connective tissue and the dermis or the facia.

As shown in FIG. 2A, targeted locations of cellulite to be treated can be marked 204 on the surface of the skin. This can be done when the patient is standing to best see cellulite. Cellulite can diminish or disappear when an individual is laying down, and should this happen, the marks 204 identify and confirm their locations (FIG. 2B).

In one aspect, local anesthetic is applied to the treatment site subcutaneously. In one approach, a long anesthesia needle (for example, 3.5 or 6 inches in length) is tunneled beyond a marked treatment site and anesthetic is administered under the marked area and along the tunneling pathway. It may be desirable to apply additional local anesthesia using a short needle transcutaneously to ensure that anesthetic extends beyond the marked target area. A distal end portion of a cellulite treatment assembly 220 is then inserted through the skin and the blunt tip is guided up into close proximity of the dermis as the tip can be tracked using the light source 352 at the distal end of the assembly as it is advanced toward septa 350 (FIG. 2C) near the marked location. Notably, the terminal end of the cellulite treatment assembly 220 in any of the disclosed embodiments can also define a tapered profile and include a tapered nosecone configured to assist in advancing the device between tissue layers. Entry sites are selected to both minimize post-operative healing time as well as to limit the use of anesthesia. The inventors have discovered given the goal of tensioning targeted septa 350, the distance from the marked location to where the treatment assembly 220 is inserted into the skin is preferably at least about 2 cm so that there is enough distance to pull and disrupt septa 350 and not have the tip of the cellulite treatment assembly exit the skin in the process. Additionally, a depth below the skin where septa 350 is preferably engaged (i.e., cut, sliced, torn, stretched, re-oriented (e.g. criss-crossing) or disrupted) is identified and determined. After determining the subcutaneous depth to be accessed for the cutting, slicing, tearing, stretching, re-orienting (e.g. criss-crossing) or disrupting of septum 350, the cellulite treatment assembly or other tool with a sharpened or blunt tip is inserted through the skin, advanced between subcutaneous tissue layers and toward septa 350. In one approach, a distal end portion of the cellulite treatment assembly is configured with a selectively activatable illuminated tip 352 that generates light (represented as illumination lines in the figures) with enough brightness to be seen through the skin. The intensity of light emitted by the tip 352 can be set to a specific constant level such that at the preferred depth below the skin for severing or otherwise engaging septa 350, the light that appears at the level of the skin as a circle or projection is of a pre-determined size. Thus, the treatment device is advanced to the target site. At the target site or as approaching the target site from the entry site, the user adjusts the depth of the tip of the treatment tool such that the circle or projection of light is the pre-determined size. Alternatively to having an illuminated tip, an illumination element can be located proximal of the treatment device 225. In this embodiment, the projection of light can be positioned under or alongside the target site such that the circle or projection of light is at the pre-determined location and the treatment element will be known to be beyond the pre-determined location. The septa 350 is tested and if confirmed as a target for treatment, the septa 350 is treated while maintaining the circle or projection at the pre-determined size. The user can also use the size of the circle or projection of light to maintain the depth of the tip of the treatment tool as it is advanced under the skin to the treatment target. In an alternative or another aspect, a sharpened tip is employed to create access to target tissue thus allowing the tool to create the desired path both into tissue as well as between tissue layers. It is expected that the depth that these tools are advanced will be between about 3 and about 10 mm below the skin surface, but it is anticipated that lesser and greater depths may also be optimal for a particular subject. It has been recognized that a more superficial treatment depth can be particularly effective in cutting all septa associated with a treatment site, as well as secondary septa are cut and can be cut in early passes within a treatment site. Accordingly, in a relative superficial approach the treatment device is advanced at a depth closer to the dermis than the superficial fascia. In any event, the depth selected is chosen for cutting, slicing, disrupting, tearing, stretching or re-orienting of the subject's septa 350. Moreover, in one embodiment, it is to be appreciated that the device 220 is formed from a substantially rigid material so that a consistent plane below the skin surface is accessed.

In another approach to precisely target and perform cellulite treatment procedures, a separate light source having a broad spread of light can be inserted into the target area through a separate incision or the same point that is less superficial to the skin layer. The treatment device is then advanced over this separate light source to identify where the treatment device is positioned through backlighting. By separating the light source from the treatment device, the treatment device can be less complicated, have fewer parts and constraints and can present a smaller profile requiring smaller incision sites and is easier to advance within tissue. Moreover, the treatment device would be subject to fewer electrical testing requirements and will potentially exhibit fewer electrical problems. In alternative or related approaches, a magnet can be incorporated into the treatment device and a magnet film can be placed over the skin to provide tracking of the treatment device. Also, a vein viewer-like apparatus can be employed to visualize reflectivity of a treatment device coated to demonstrate high reflectivity to identify device location, or near-IR light is employed to detect temperature differences between the treatment device and the patient's skin to thus identify treatment device positioning.

Using palpation, direct visualization (for example, transillumination or endoscopic) or non-invasive visualization (for example, ultrasound or fluoroscopic) or other means for determining the position of the interventional tool such as markings along the length of the instruments and its path within tissue, or providing the interventional instrumentation with radiopaque markers, the tool is placed at a site below where cellulite (for example a dimple) is seen on the subject's skin. The treatment device is advanced through septa 350 and to where the treatment device 225 is in a position best suited to accomplish the identification of target septa and the cellulite removal or minimization treatment. As shown in FIGS. 2D-E, in one approach, the treatment device 225 is passed beyond septa 350 in a stored or collapsed configuration, and once positioned beyond septa 350, a hook is deployed. Next, the tool assembly 220 is then pulled proximally to tension septa 350, such as by hooking the septa (FIG. 2F). In another approach, the treatment device 225 is passed a few millimeters lateral, preferably about 1 to about 10 millimeters, more preferably about 3 to about 6 millimeters, and beyond the target location, a hook is deployed and then swept laterally toward the target followed by pulling proximally to hook and tension septa. In one aspect, during initial deployment, the hook structure defines a relatively flat angle, that is, an edge of the hook is at about 80 degrees relative to a long axis of the elongate member 224, which results in providing the treatment instrument with substantial reach. Once it is time to cut or otherwise engage septa 350, the treatment device 225 is manipulated so that its blade or other cutting surface is exposed at a more steep angle better for cutting, such as about 70 degrees relative to the long axis of the elongate member 224. During these and other steps, transillumination can be employed to track the treatment device and guide the procedure. The marks 204 can facilitate targeting of septa 350 while using transillumination to see the location of the treatment device 225. In other approaches, a separate device can be employed to engage septa 350 to see if such septa are the source of a dimple or depression expressed on the outside of the skin. Such a secondary device can be placed remotely from the target (i.e. lesion) and configured to be capable of applying tension to the surface of skin in a predetermined direction so as to create the effect of gravity and produce the visualization of the lesions while the patient is in a prone position (i.e. a broad region of adhesive attached to a spring mechanism such that a predetermined force would be applied relatively parallel to the surface of the skin in the direction the skin would move when standing in gravity). Using this additional device could further help the confirmation and location of lesions and allow confirmation that the treatment was effective. Also, in various approaches, a portion of the elongate member can be configured to transition from a smaller state to a wider or larger state, wherein in the wider or larger state a cutting surface (i.e. sharpened edge or energy) is presented to cut tissue, the device being sized and shaped to be inserted through the skin and engage one or more regions of septa subcutaneously.

It is noted that septa causing a dimple or depression may be coming from various angles and locations relative to the dimple or depression seen on the skin rather than being directly below the dimple or depression, and may be due to one or only a few septa or a large number of septa that remotely cause the depression or dimple. Thus, so engaging certain septa will be reflected in some change in the dimple or depression on the skin. A determination is made concerning the correspondence with marks 204 made on the skin and the dimples being formed or re-formed. If the initial septa 350 that the user presses on or pulls on using the tool do not recreate a dimple or depression in the marked area 204, then the user releases those initial septa that were engaged and repositions the tool at different septa and presses on or pulls again. This is repeated until the septa responsible for a dimple or depression in the marked location are identified (FIG. 2F). Once proper septa are identified, the tool 225 is manipulated to cut, slice, disrupt, re-orient, stretch or tear septum 350 connecting tissue layers. In one approach, a blade 353 is deployed and presented for treatment.

After the proper septa have been severed, disrupted, stretched, or re-oriented, the treatment element 225 is moved back to its initial collapsed configuration. The treatment element is then advanced beyond the marked treatment location, the treatment element (e.g., hook) is deployed and then pulled back under the marked treatment location to confirm that all of the septa responsible for causing the marked dimple or depression have been separated intra-operatively. Again, multiple passes are taken to ensure that all cellulite-creating septa, including secondary septa are cut. If they have not been, the tool is manipulated to sever, disrupt, stretch, or re-orient additional septa. An approach is taken working right to left through an entire marked margin area and the steps are repeated until all of the septa responsible for creating the marked dimple or depression have been severed or sufficiently stretched and the dimple or depression cannot be re-created intra-operatively using the tool. Alternatively, or additionally, to check that the marked treatment targets have been adequately separated, treatment can be conducted with the patient lying down and the patient can then be asked to stand up off of the procedure table to let gravity act on the body to see whether marked treatment areas have been treated. Where the patient is asked to stand, steps are taken to maintain a sterile field and appropriate draping is provided to the patient. Where necessary, further treatment can be conducted on the unresolved areas. Such manipulation results in selective rupture, tearing, cutting or slicing of targeted septum 350, and the removal or minimization of dimples and the appearance of cellulite on skin (FIG. 2G). Thereafter, the treatment element (e.g., hook and/or blade) is retracted back in and the tool 220 is removed from the site to be withdrawn from the body or repositioned in any direction along and within the target tissue plane to treat additional areas.

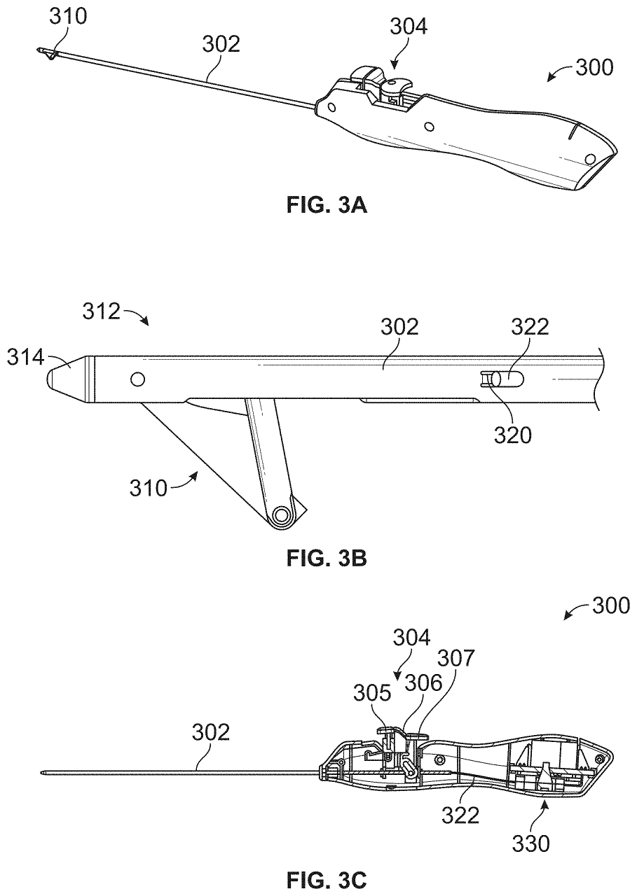

With reference now to FIGS. 3A-C, there is shown a treatment system that embodies certain functionality and a selected number of features described above. The treatment system includes a handle assembly 300 from which an elongate member 302 extends. The handle assembly 300 defines a contoured profile sized and shaped to conveniently fit within an operator's hand. Various actuating members 304 are provided on the handle assembly 300 the manipulation of which accomplishes the deployment of a treatment device 310 employed to engage, test and/or cut septa. The elongate member 302 has a length and cross-sectional profile configured to be placed between tissue layers and to be advanced and extend to septa existing in a target treatment area. A distal end 312 of the treatment system (FIG. 3B) includes the treatment device 310 and a terminal end 314.

The distal end 312 portion (FIG. 3B) of the treatment system is also equipped with an exit port 320 for a light fiber 322 that provides the transillumination functionality described above. As shown in the cross-sectional view of the treatment system (FIG. 3C), the light fiber 322 extends distally from a light energy source and focusing assembly 330 configured within the handle assembly 300. The light fiber 322 extends distally through the handle and within the elongate member 302 to the exit port 320. A battery provides energy to an LED which generates light energy.

Various further alternative approaches to cellulite treatment systems can be employed. With reference again to FIGS. 3B-C, the actuating members 304 are provided on the handle assembly 300 the manipulation of which accomplishes the deployment of a treatment device 310 employed to engage, test and/or cut septa. Thus, manipulating the actuating members 304 results in the treatment device 310 assuming home or sheath, open or hook and active or cut configurations. In use, the actuating members 304 are in a first proximal position and the treatment device 310 is in the home or sheath position (See also FIG. 2C). Next, the actuating members are advanced distally by pushing on the slider 306 so that the treatment device 310 assumes an open or hook configuration (FIG. 2E) and thereafter, an active button 307 of the actuating members is depressed so that the treatment device defines an active or cut configuration (FIG. 2F-G). To return to home, the home button 305 of the actuating members 304 is manipulated so that the treatment device 310 returns to the home or sheath configuration.

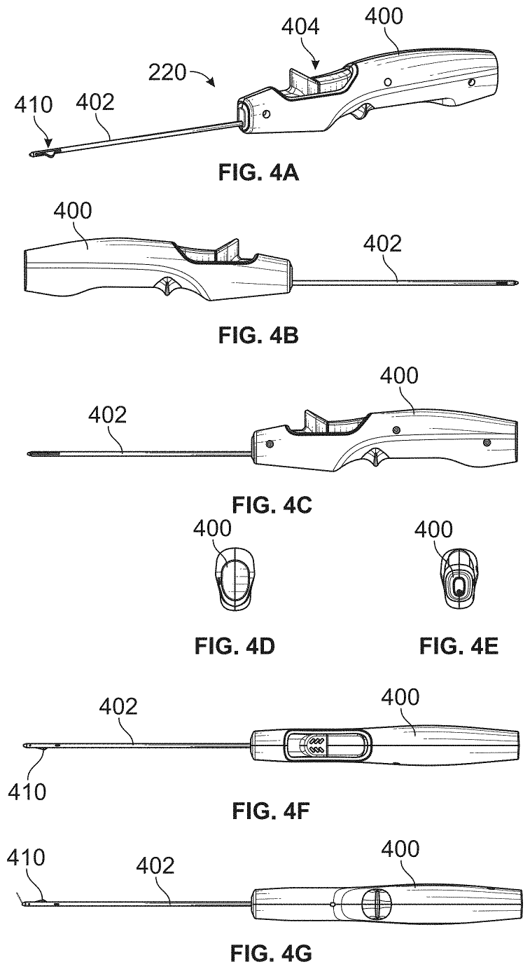

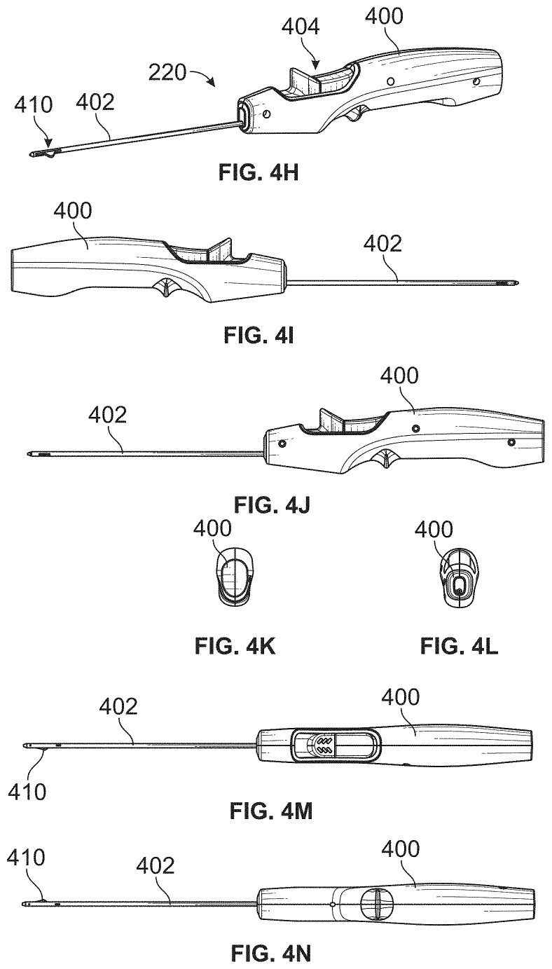

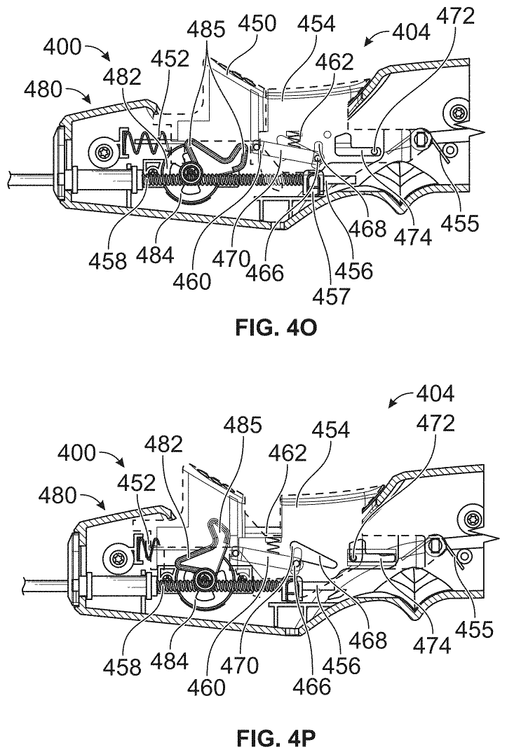

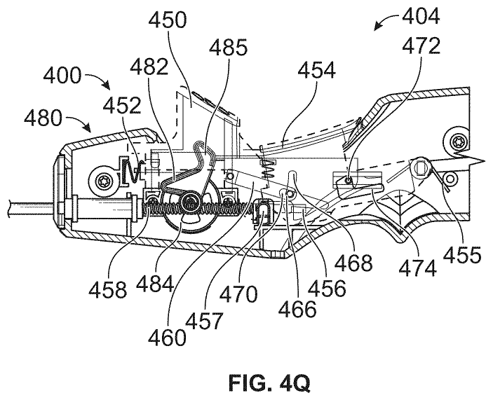

Turning to FIGS. 4A-N, there are shown various views depicting among other things, the ornamental design of alternative approaches to a cellulite treatment assembly 220. The treatment assembly 220 includes a handle assembly 400 from which an elongate member 402 extends. Here also, the handle assembly 400 defines a contoured profile sized and shaped to conveniently fit within an operator's hand. Moreover, an actuation assembly 404 is provided on the handle assembly 400 the manipulation of which accomplishes the deployment of a treatment device 410 employed to engage, test and/or cut septa. Again, the elongate member 402 has a length and cross-sectional profile configured to be placed between tissue layers and to be advanced and extend to septa existing in a target treatment area, and a distal end portion of the treatment system includes the treatment device 410. As with the previously described embodiment, the distal end portion of the treatment system can also be equipped with an exit port for a battery powered light that provides the transillumination functionality described above (See also FIG. 3C).

With reference to FIGS. 40-Q, there is shown a cross-sectional view of the handles depicted in FIGS. 4A-N. This approach to a handle assembly is configured to reduce the number of steps or actions required by a user to operate a treatment device. By doing so, potential for user fatigue is diminished and an amount of time required for a user to learn and internalize how to use the treatment device can be reduced. Handle assemblies can function by having three states, namely, home, open or hook and active or cut, where a user must take an action in order to place the device into a next state such as by pushing a button or moving a slider. In certain approaches to handles, the user must take an action to return the handle assembly to a home position. However, in the presently described embodiment, the user action associated with returning the handle assembly to the home state can be eliminated. In a specific approach, the return of the handle assembly to the home position can be made despite tension provided by tissue against the treatment device. This may be necessary when a user has completed a cut, and a blade of the treatment device comes to rest against nearby tissue structures with some amount of tension that can interfere with the mechanism returning to the home position. Eliminating the manual action to return to home also reduces the number of buttons or sliders presented to the user and as stated, facilitates more quickly learning how to use the treatment device. Further, the disclosed handle assembly prohibits an actuating mechanism to return to the home position until a tissue cutting actuating mechanism is released. This can prevent confusion as to which state the device currently assumes.

As shown in FIGS. 40-Q, the handle assembly 400 includes a body supporting an actuating assembly 404. The actuating assembly includes a slider 450 biased by a spring 452, and a cut button 454 also biased by a spring 455. The slider 450 and cut button 454 are configured to manipulate a pushrod 456 including a pushrod block 457, that is also biased by a spring 458. The pushrod 456 is operatively associated with a treatment device (not shown). The pushrod 456 is not permanently connected to either the slider 450 or the cut button 454, but is handed off between the two structures. Once the cut button 454 is released, the pushrod 456 is freed from the slider 450 and cut button 454, thereby allowing a full potential energy of its spring 458 to work against any device residual tip tension without being hampered by friction imposed by the mechanical action of handle components. In this way, performance against tissue tension at a distal tip of the treatment device can be optimized. Action of the pushrod 456 is controlled by a latch 460 that is loaded by a spring 462. The latch 460 includes a first projection 466 that rides in a first track 468 formed in the slider 450 and a second triangular shaped track 470 formed in the cut button 454, and a second projection 472 that rides in a third track 474 formed in the cut button 454.

Also configured within the handle assembly 400 is a damper assembly 480. The damper assembly 480 includes a damper arm 482 and one or more dampers 484. Although the component parts of the damper assembly can assume various constructions, as shown, the damper arm 482 includes diverging projections 485 and is configured to be pivotable within the handle 400 relative to the damper 484. The damper 484 includes a disc-like portion 486 that functions like a brake as it engages structure of the damper arm 482. The damper assembly 480 is operatively associated with the slider 450 to control the return of the handle 400 to a home state. The damper assembly 480 slows the return of the slider 450 to a proximal position to provide a slow close feature or functionality. The damper assembly 400 also reduces noise associated with the return of the slider 450 to a home position. In this way, a treatment procedure involving this damper feature is more controlled and facilitates avoiding startling a patient with noises emanating from the treatment device during a procedure.

In a home state (FIG. 4O) where the treatment device is in a stowed position, the slider 450 is in a proximal position and the cut button 454 is undepressed. As the slider 450 is pushed forward, the first projection 466 travels within the first track 468 and locks the device in an open state (FIG. 4P). The slider 450 is advanced distally against the force provided by springs 452 and 458. Such action causes the slider 450 to engage a damper arm projection 485 and to rotate the damper arm 482 through its engagement with the damper 484. In this way, the slider 450 is provided with a smooth transition to its distally locked position. This advancement of the slider 450 also causes the pushrod 456 to be advanced against the spring 458 associated therewith. The translational force asserted by the user against the slider should be optimized to be between 0 and 4 pounds, to reduce fatigue and provide a comfortable experience.

At this point, the treatment device is deployed to present a hook configuration but a blade or cutter of the treatment device is covered or undeployed. Next, when the cut button 454 is depressed (FIG. 4Q), the first projection 466 unlatches from the first track 468 but the treatment device is held in the open state by the second projection 472 configured in the third track 474 formed in the cut button 454 as well as the cut button 454 against the pushrod block 457. Now, the treatment device is in a cutting configuration where the blade or cutter of the treatment device is exposed and can be used to treat target tissue.

Upon releasing the cut button 454, the second projection 472 unlatches, allowing the slider 450 to return to its proximal position and the cut button 454 to return to its undepressed configuration. The return of the slider 450 to its home position is facilitated by the energy provided by the spring 452 associated with the slider 450. The damper assembly 480 slows and quiets the return action of the slider 450 through the engagement of the damper arm 482 with the damper 484. Such control is provided when the slider 450 engages another projection 485 to rotate the damper arm 482 through its engagement with the damper 484, and frictional forces slows the action of the slider 450. The treatment device thus automatically returns to the stowed configuration and the handle assembly 400 is in the home state (FIG. 4O).

After completing treatment of one target area employing one or more of the disclosed treatment devices, the procedures described herein can repeated to treat other target areas. Accordingly, the same devices can be employed to access tissue layers below other sites or depressions existing in skin. Notably, in one embodiment, the devices are capable of anesthetic delivery as needed or desired when progressing to additional or new locations. There is thus provided a system configured to treat all target areas on the body through a limited number of small entry sites, including through a single entry site on each side of the patient. It is to be recognized that the system can further include structure permitting the assembly to be steerable to subcutaneous treatment sites. In such an embodiment, the device would be configured to define longitudinally flexible material, and the instrumentation would be steered to the desired position within tissue. Moreover, in certain applications, the device has a stiffness that varies along its length. In another embodiment, the treatment and fat collection devices are embodied in deflectable catheters.

In each of the disclosed approaches and apparatus, should engagement of such septa result in some change in the dimple or depression expressed on the skin, the treatment structures are manipulated to disrupt, cut or slice the tissue. Thus, the treatment structures are opened and tissue is placed between its cutting structure. Next, the cutting structures are advanced against or caused to be closed about the tissue to thereby cut, slice or sever the tissue, thus relieving the tension between tissue layers and eliminating or minimizing the appearance of the unwanted feature on the skin. Actuation is accomplished from a proximal end of the treatment device such as by pulling a wire or advancing and pushing an elongate member associated with the scissor arrangement. Illumination can be provided by a light configured proximal of the treatment or fat collection devices so that transillumination can be employed to track the location of the distal portion of the treatment assembly. Additionally, or alternatively, in each disclosed embodiment, illumination can be via a lightguide from an external light source or via one or more LEDs. Illumination aids the user both with locating the treatment device as well as proper depth placement as transillumination decreases with increasing tool depth. In one aspect, the amount of illumination is set to ensure proper depth of a treatment device or structure, the level of illumination targeted being adjusted for skin type, thickness, presence of fat and pigment.

In employing one or more of the disclosed embodiments in a treatment procedure, there is an expectation that there are instances where it is preferable to not disrupt a hooked tissue, and in such a case it is desirable to release or disengage the hooked tissue. In certain approaches, to release or disengage, the treatment device would be advanced or twisted away from the hooked tissue. It is thus recognized that a challenge exists in that there may be additional tissue in the area which could be unintentionally re-engaged by the treatment device when it is in a hooking configuration, and stowing of the treatment device may be inhibited by adjacent patient anatomy.

Accordingly, various approaches to aesthetic treatment methods and apparatus are presented. The disclosed approaches are configured to provide an effective and focused approach to treating, minimizing and preventing unwanted skin features. The disclosed approaches can also be used to repair and reduce the appearance of unwanted features in a targeted manner. Further, the disclosed proactive treatment modalities are easy and effective to use.

Some of the specific aspects of the present disclosure include one or more of focal treatment of just the tissue responsible for causing unwanted features in the skin; minimizing bruising; accessing all treatment targets from limited, cosmetically acceptable entries; capture and retention of tissue while separating the tissue; intra-operative confirmation of the treated target; needle-diameter sized tools for small openings; and transillumination identification of tool tip location.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the present disclosure.

That which is claimed is:

1. An aesthetic treatment system for treating appearances of a patient's skin associated with a treatment site, comprising:
an aesthetic treatment assembly including an elongate member extending from a handle and configured to be insertable through the skin and a mechanical connective tissue disrupting element attached to a distal end of the elongate member that is capable of expanding at least one region from a smaller state to a wider state and engaging one or more regions of connective tissue subcutaneously, wherein the mechanical connective tissue disrupting element is configurable to both define connective tissue hooking structure as well as connective tissue severing or disrupting structure;
wherein the handle includes a body and an actuating assembly configured to control operation of the mechanical connective tissue disrupting element, the actuating assembly including actuating structure that places the mechanical connective tissue disrupting element into connective tissue severing or disrupting structure and facilitates an automatic return of the mechanical connective tissue disrupting element to a home position.

2. The aesthetic treatment system of claim 1, wherein the actuating assembly includes a slider and a cut button.

3. The aesthetic treatment system of claim 2, wherein the slider is configured so that advancement thereof relative to the body of the handle places the mechanical connective tissue disrupting element into an open state to present a hook configuration.

4. The aesthetic treatment system of claim 3, the handle further comprising a damper assembly that is configured to one or more reduce noise or speed associated with the handle returning to a home configuration.

5. The aesthetic treatment system of claim 4, wherein the damper assembly includes a damper configured to engage a damper arm, the damper arm being responsive to movement of the slider.

6. The aesthetic treatment system of claim 1, wherein the handle defines a contoured profile sized and shaped to conveniently fit within an operator's hand
